(19) United States
(12) Patent Application Publication
(10) Pub. No.: US 2021/0162230 A1
(43) Pub. Date: Jun. 3, 2021

(54) Accoutrement with Ion Emitters

(71) Applicant: Yat Fai WONG, VANCOUVER (CA)

(72) Inventor: Yat Fai WONG, VANCOUVER (CA)

(21) Appl. No.: 16/700,753

(22) Filed: Dec. 2, 2019

(51) Int. Cl. A61N 2/02; A61N 2/00; A41D 13/12

(52) U.S. Cl. CPC A61N 2/02; A61N 1/0484; A41D 13/1281; A61N 2/004

(57) ABSTRACT

An accoutrement with electromagnetic field generator is disclosed. The invention is comprised of four main parts. A first part being an accoutrement (such as a vest, jacket, hat etc.); a second part being micro controller that can be ‘paired’ with a mobile computing device (such as a cell phone, tablet etc.) embedded inside said accoutrement; a third part being a plurality of hexagonal-shaped, electromagnetic, resonant frequency coils (referred to herein as ‘coils’)—also embedded inside said accoutrement; and a mobile device software application (app) with an archive of digital therapy sound files that users can choose from. These therapy files are ported through said coils allowing users to receive a plurality of healing, electromagnetic fields and ions. Upon receiving sound file frequencies, said copper winding inside the coils conducting an electromagnetic field and emitting ions to be received by a user seeking therapeutic benefits.

### FIELD OF THE INVENTION

The present invention generally relates to resonant frequencies. More specifically, it relates to a method and system for generating electromagnetic fields from amplified, digital frequencies from a mobile computing device over a users' upper torso.

### BACKGROUND

Resonant frequency therapy has been in use since the invention of electricity in the late 1800s. Pulsed electromagnetic fields (known as PEMF) was widely adopted in East and Western Europe but its use was restricted to animals in North America until. Veterinarians became the first health professionals to use PEMF therapy, usually to heal broken legs in racehorses, and by a scientist known as Royal Rife. Royal worked alongside Carl Zeiss in the microscope industry and conducted research in microscoping lighting frequencies. Using microscopes, he transmitted various frequency waves with a microscope device and targeted known pathogens. In tests using B. coli (bacillus coli) Rife found that the bacteria, if exposed to radio-frequency energy at certain frequencies, would “deactivate”, or die. Later testing showed the same results with other samples of B. coli at the same frequencies. One of the original PEMF devices consisted of a Helmholtz coil which generated a magnetic field. The patient's body was placed inside the magnetic field to deliver treatment. In the 1950s, researchers began extrapolating these frequencies to treat human diseases and Dr. Abraham Ginsberg successfully treated patients who had various infections and went on to found the Ginsberg Foundation for Medical Research Symposium.

During the 1970s, a medical researcher named Bassett introduced a new approach for the treatment of delayed fractures. This technique employed a very low frequency signal to be applied for non-union, delayed fractures. The use of electrical stimulation in the lumbosacral region was first attempted by Alan Dwyer of Australia. In 1974, he reported successful initiation of graft incorporation in 11 of 12 fusion patients. In 2004, a pulsed electromagnetic field system was approved by the Food and Drug Administration (FDA) as an adjunct to cervical fusion surgery in patients at high risk for non-fusion. In 2009, a majority of PEMF wellness devices resembled a typical yoga mat in dimensions but were slightly thicker and housed several, flat, spiral coils to produce an even electromagnetic field. Frequency generators are often used to energize the coils to create a pulsed electromagnetic field. The majority of PEMFs were manufactured in Germany, Austria and Switzerland and were imported into North America as electric massagers or full body electric yoga mats. They were either placed on a massage table for clinical use or directly on the floor in the home to practice simple yoga postures. A wide variety of professional and consumer PEMF devices are sold and marketed as FDA registered wellness devices.

Electromagnetism has also been found to play a role in plant growth. In 1862, Louis Pasteur discovered that magnetism affected the growth of plants while experimenting with fermentation. He found that magnetism had a significant effect on the growth of plants and that such a treatment could be used effectively in agriculture by exposing seeds, water, soil and nutrients in the soil. This strategy is now used for agricultural development in many parts of the world. Later in the 1900s, researchers began experimenting with electromagnetism and seeds. U.S. Pat. No. 4,020,590 was issued to Albert R. Davis in for the apparatus and method of treating seeds with the magnetizer. The process of magnetizing seeds substantially accelerated the growth, and improved the quality of plants. The process of magnetizing water also has benefits to plants as it increases its solubility and filtering ability. Nutrients are dissolved easily in magnetized water, which can then be used for irrigation to increase the fertility of the soil. Magnetized water helps plants accumulate minerals in the water. This efficiency decreases the amount of water needed for irrigation. Fertilizers are also more easily dissolved in magnetized water and penetrate into the plant cell, thereby decreasing the amount of fertilizers required.

Electromagnetic stimulation during the fermentation of wines and alcoholic beverages has also had positive results. A French researcher named Matter found that continuously supplying low-power electromagnetic stimulation in the range of 20-30 kHz to S. cerevisiae (wine yeast) could enhance ethanol production. The optimum ultrasonic treatment at the frequency of 25 kHz resulted in 15.6% ethanol concentration that was higher than in the control system. Researchers at the University of Bologna have also demonstrated that the use of PEF can enhance the extraction of phenolic compounds in wine and increase color intensity in red wines. In fact, color stability was also assisted by the extraction of valuable polyphenolic compounds, monomers (flavonoids, hydroxycinnamic acids, tannins) and polymers (condensed tannins from grape skin) that occur during such exposure. The main mechanism responsible for the increasing mass transfer was the electroporation of vegetable cell membranes, which also enabled to reduce the maceration process, and to enhance the extraction of bioactive compounds from grape using PEMF.

In 2015, the FDA reclassified PEMF devices from the Class 3 category (insufficient safety information) to a Class 2 status (approved with safety measures). PEMF devices that have been FDA cleared to make health claims require a doctor's prescription for use. [U.S. Pat. No. 5,402,782, issued Apr. 4, 1995, and U.S. Pat. No. 5,553,610, issued Sep. 10, 1996, both to Robert A. Lodder disclosed a small magnetic, near-infrared radiation and an acoustic wave delivery system for therapeutic purposes. U.S. Pat. No. 4,535,775 disclosed a method of healing bone fractures by applying to electrodes coupled to the skin of a living body in the vicinity of a bone fracture and transmitting an electromagnetic frequency in the range of 20-100 KHZ and a value in the range from about 2 to 10 volts. U.S. Pat. No. 9,812,246B1 granted to Nunez disclosed a toroid frequency coil, but it is not part of a larger treatment system. Several commercially available rife machines are available on the market that deliver electromagnetic frequencies using attachable electrodes, passive electrostatic tubes and hand held cylinders. However, none were found in the prior art that incorporated a coil bound in a multi-point pattern connected to external amplifiers and digital audio frequency files for mobile devices. In addition to the benefits of frequency coils, it is well-known that the addition of gem stones within such coils can have therapeutic benefits (known as piezoelectric or pyro-electric effects as additional ions are generated from the coil. Some of the more important benefits from ions is that they clear the air of airborne allergens such as pollen, mold spores, bacteria and viruses. Besides they also clear the air of dust, pet dander and cigarette smoke.

### SUMMARY OF THE INVENTION

The device herein disclosed and described provides a solution to the shortcomings in the prior art through the disclosure of a method and system for transmitting resonant frequencies through portable, hexagaonal-shaped, copper coil emitters embedded inside an accoutrement. The coils are wound with copper wire and are connected to a micro controller which in turn is paired to digital audio frequency library on a mobile device using BlueTooth™. When activated, audio files are played into the amplifier where they frequencies are amplified and sent to the ion plugin allowing it to emit an ‘electromagnetic vortex field.’

An object of the invention is to provide a means to deliver electromagnetic frequencies to specific areas of the user's body. The user plays an audio file on their mobile device, it sends the frequency to the coils inside the ion accouterment and then rotates the signal at the speed of light in a circular fashion creating a frequency pulsed electromagnetic vortex field with a plurality of positive ions. The coils are located within the accoutrement at positions the unit within close proximity to vital organs of the body.

Another object of the invention is to provide a means to allow a user adjust the level of electromagnetic frequencies delivered. The user can use a setting on the software app to adjust the amount of frequencies that are emitted from the device depending on the therapy needed.

Another object of the invention is to provide a means to allow different types of coils to be mounted to be used in the accoutrement. Users can select from a wide array of manufactured coils to use depending on the condition they would like to treat.

It is briefly noted that upon a reading this disclosure, those skilled in the art will recognize various means for carrying out these intended features of the invention. As such it is to be understood that other methods, applications and systems adapted to the task may be configured to carry out these features and are therefore considered to be within the scope and intent of the present invention, and are anticipated. With respect to the above description, before explaining at least one preferred embodiment of the herein disclosed invention in detail, it is to be understood that the invention is not limited in its application to the details of construction and to the arrangement of the components in the following description or illustrated in the drawings. The invention herein described is capable of other embodiments and of being practiced and carried out in various ways which will be obvious to those skilled in the art. Also, it is to be understood that the phraseology and terminology employed herein are for the purpose of description and should not be regarded as limiting.

As such, those skilled in the art will appreciate that the conception upon which this disclosure is based may readily be utilized as a basis for designing of other structures, methods and systems for carrying out the several purposes of the present disclosed device. It is important, therefore, that the claims be regarded as including such equivalent construction and methodology insofar as they do not depart from the spirit and scope of the present invention. As used in the claims to describe the various inventive aspects and embodiments, “comprising” means including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The objects features, and advantages of the present invention, as well as the advantages thereof over existing prior art, which will become apparent from the description to follow, are accomplished by the improvements described in this specification and hereinafter described in the following detailed description which fully discloses the invention, but should not be considered as placing limitations thereon.

### BRIEF DESCRIPTION OF THE FIGURES

The accompanying drawings, which are incorporated herein and form a part of the specification, illustrate some, but not the only or exclusive, examples of embodiments and/or features.

FIG. 1 shows a perspective view of the invention.

FIG. 2 shows a perspective view of the invention's internal components.

FIG. 3 showing the invention's software application.

Other aspects of the present invention shall be more readily understood when considered in conjunction with the accompanying drawings, and the following detailed description, neither of which should be considered limiting.

### DETAILED DESCRIPTION OF FIGURES

In this description, the directional prepositions of up, upwardly, down, downwardly, front, back, top, upper, bottom, lower, left, right and other such terms refer to the device as it is oriented and appears in the drawings and are used for convenience only; they are not intended to be limiting or to imply that the device has to be used or positioned in any particular orientation.

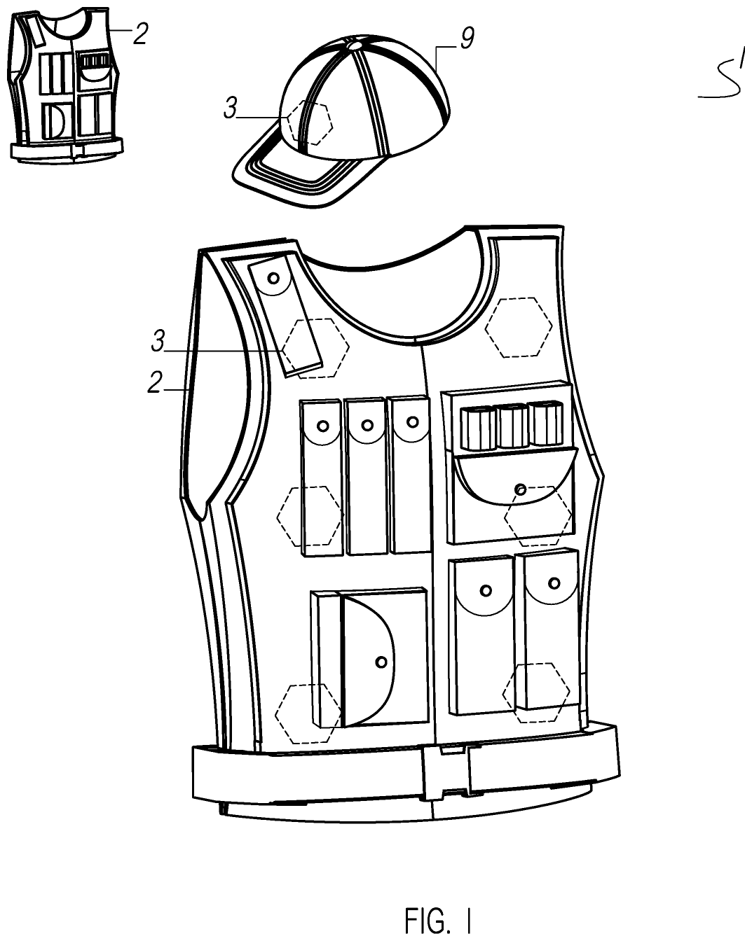

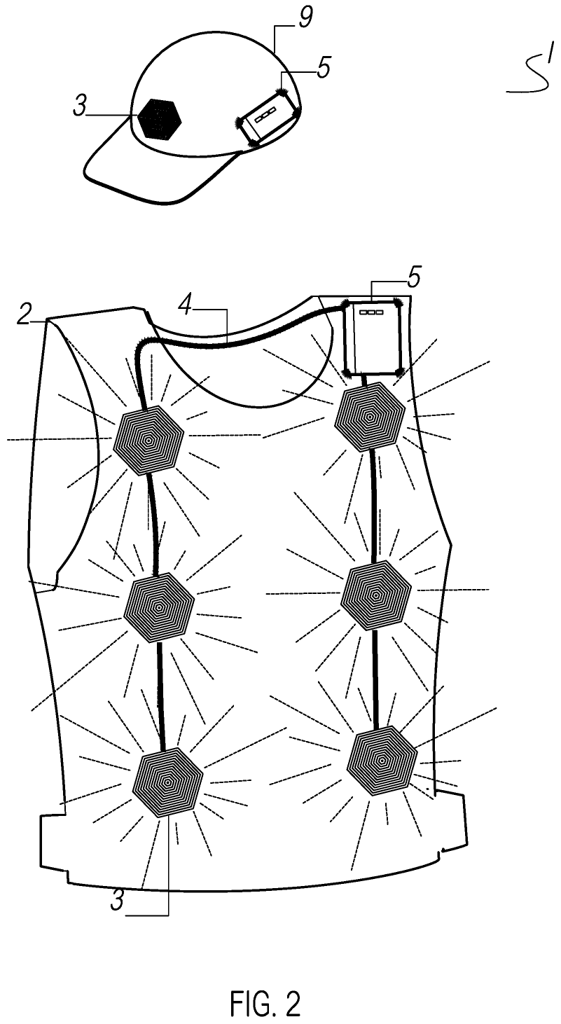

FIG. 1 showing a perspective view of an embodiment of the invention 1 having a plurality of coils 3 inside a vest 2 and hat 9—other embodiments may include other accoutrements such as but not limited to jackets, sweaters and the like. The figure showing coils 3 having wire windings configured in a hexagonal—shape and comprised of, but not limited to, a conductive metal such as copper etc. Said coils 3 being disposed and embedded proximally inside accoutrement 2. FIG. 2 showing the coils 3 having at least three levels of windings and all being interconnected to microcontroller 5 by means of wiring harness 4. Said micro controller 5 being powered by an onboard battery such as but not limited to lithium ion, nickel hydride and the like and having an onboard BlueTooth™ receiver to transmit audio signals from a mobile device and send said signals to coils 3 simultaneously. FIG. 2 also showing invention 1 in use with its electromagnetic vortex field being emitted from the metal wire coils 3 and rotating a signal at the speed of light in a circular fashion creating a frequency pulsed electromagnetic field vortex field with a plurality of positive ions that surrounds a user. Other embodiments of the invention 1 having a stone, including but not limited to gems, sapphires, diamonds and the like being disposed proximally on said coils 3 to enhance frequency intensities.

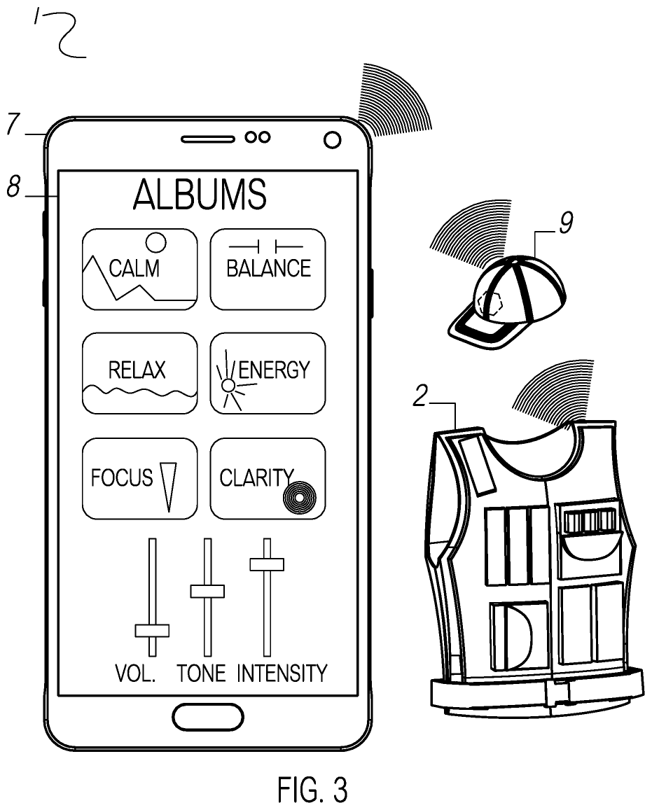

FIG. 3 showing the invention's software application 8 being operated on a mobile device 7 (such as but not limited to cell phones, tablets, and computers etc.) and having sound file selections that allow users to determine which types of audio frequencies are transmitted to coils 3. Said audio files being comprised of a plurality of ‘Life Force’ frequencies associated known to be associated with mitigating a multitude of maladies such as, but not limited to: Allergies, emotional issues, chronic illnesses etc. Frequencies may include but not be limited to: 1 hz to 30,000 hz at a multitude of incremental frequencies preset by said digital audio devices. Said software application 8 having a multitude of setting options such as but not limited to audio volume, tone and intensity etc. Some embodiments of said software application 8 may be purchased by users as part of a subscription and compatible with existing app services including but not limited to Google Play, Apple etc.

Other embodiments of invention may have a standalone base station sending audio content to the device without the need for a user's mobile computing device. Said base station being comprised of a proprietary digital audio player configured to perform a varietal of functions such as but not limited to controlling digital file intensity, volume, frequency, tone and the like. The base station also having a software memory to hold a multitude of proprietary digital audio files therein as well as a small user interface screen. Said invention software being written from code that may include, but not be limited to: Java, C++™, Visual Basic™, Fortran™, Basic™ and the like. The software also compatible with a plurality of operating systems such as, but not limited to: Windows™, Apple™, and Android™, and compatible with a multitude of hardware platforms such as, but not limited to: personal desktops, laptops, tablets, smartphones and the like.

It is additionally noted and anticipated that although the device is shown in its most simple form, various components and aspects of the device may be differently shaped or slightly modified when forming the invention herein. As such those skilled in the art will appreciate the descriptions and depictions set forth in this disclosure or merely meant to portray examples of preferred modes within the overall scope and intent of the invention, and are not to be considered limiting in any manner. While all of the fundamental characteristics and features of the invention have been shown and described herein, with reference to particular embodiments thereof, a latitude of modification, various changes and substitutions are intended in the foregoing disclosure and it will be apparent that in some instances, some features of the invention may be employed without a corresponding use of other features without departing from the scope of the invention as set forth. It should also be understood that various substitutions, modifications, and variations may be made by those skilled in the art without departing from the spirit or scope of the invention.

What is claimed is:

1. A system to deliver electromagnetic fields to the body. The system comprised of the following parts:
(a) an accoutrement;
(b) electromagnetic, resonant frequency coils;
(c) a micro controller; and
(d) a mobile device software application.

2. The system to deliver electromagnetic fields to the body of claim 1 wherein the delivering being performed by the electromagnetic, resonant frequency coils emitting frequencies.

3. The delivering of frequencies of claim 2 wherein the frequencies are comprised of audio signals.

4. The system to deliver electromagnetic fields to the body of claim 1 wherein the electromagnetic, resonant frequency coils are embedded into the accoutrement.

5. The system to deliver electromagnetic fields to the body of claim 1 wherein the electromagnetic, resonant frequency coils are connected to the micro controller with a wiring harness.

6. The system to deliver electromagnetic fields to the body of claim 1 wherein the micro controller receiving audio signals from the mobile device software application and sending them to the electromagnetic, resonant frequency coils.

7. The system to deliver electromagnetic fields to the body of claim 1 wherein the electromagnetic, resonant frequency coils are embedded into the accoutrement.

8. The system to deliver electromagnetic fields to the body of claim 1 wherein the mobile device software application allowing users to adjust audio volume, tone and intensity of the audio signals of claim 3.
